(19) United States
(12) Patent Application Publication
(10) Pub. No.: US 2005/0019900 A1
(43) Pub. Date: Jan. 27, 2005

(54) Device for the capacitive measuring of a fill level

(76) Inventors: Patrick Broyer, Villeurbanne (FR); Pierre Imbaud, Pommiers (FR)

Correspondence Address: OLIFF & BERRIDGE, PLC, P.O. BOX 19928, ALEXANDRIA, VA, 22320, US

(21) Appl. No.: 10/498,810

(22) PCT Filed: Dec. 18, 2002
(86) PCT No.: PCT/FR02/04414

(30) Foreign Application Priority Data
Dec. 19, 2001 (FR) 01/16439

(51) Int. Cl. C12M 1/34; G01N 11/00; G01N 33/487

(52) U.S. Cl. 435/287.1; 73/53.01

(57) ABSTRACT

A method of controlling the presence or absence of a volume of liquid in a container or of controlling the filling of a container with a liquid volume. A channel passes through the container and channel opens into a shaft which collects all or part of the liquid. The channel(s) and shaft(s) form a fluidic network. Implementation of the method includes: detecting the capacity, or another related parameter, such as the relative permittivity of the air present in the container, the oscillation frequency or the voltage, at each shaft before the liquid is injected; detecting the capacity, or another related parameter, at each shaft after the liquid has been injected; determining the capacitive variation, or a variation in a related parameter, at each shaft; and deducing the quantity of liquid present at each reaction shaft. The invention is particularly suitable for use in the field of diagnosis.

The present invention relates to a method for monitoring the presence or absence of a volume of liquid in a container or for monitoring the filling of a container with a volume of liquid, with at least one channel which leads to at least one well that receives some or all of the liquid passing through the container, the channel or channels and the well or wells constituting a fluidic network. The invention also provides the implementation of the method mentioned above for monitoring the presence or absence of a volume of liquid in a container or monitoring the filling of a container with a volume of liquid. It also concerns the implementation of the method referred to above for counting microorganisms present in the initial volume of liquid.

The present invention furthermore discloses a device for monitoring the presence or absence of a volume of liquid in a container or for monitoring the filling of a container with a volume of liquid, implementing the method mentioned above, with at least one channel which leads to at least one well that receives some or all of the liquid passing through the container, the channel or channels and the well or wells constituting a fluidic network. The invention also divulges the use of this device for monitoring the presence or absence of a volume of liquid in a container or monitoring the filling of a container with a volume of liquid. More precisely, this use of the device may be applied to counting the microorganisms present in the initial volume of liquid.

The prior art consists of documents which pertain to various fields. The first field concerns measurement by direct contact between an electrode and the liquid. A representative example consists of the capacitive detection with the aid of conductive cones, for example made of carbon, which are used on tri-axial automata and make it possible to detect liquid in a tube. This is, for example, the case with Patent Application EP-A-0 341 438. The position of the interphase of an electrically conductive fluid may furthermore be detected by means of a capacitive probe. This detection is based on the existence of a current between the probe and the liquid, the electrical capacitance measured by the probe being affected by the variation in liquid level. Such a probe may, in particular, include a central metal rod clad with an insulating plastic material, for example applied by heat-shrinkage, which is immersed in the conductive liquid to a varying degree. The variation of the inter-electrode capacitance, between the clad rod and the conductive liquid, resulting from the variation in the liquid level is then measured as is the case in Patent Application FR-A-2 772 477.

This technical field is not relevant because it requires direct contact between the electrode, consisting of the cone or the probe, and the liquid. The drawbacks of such a technique are principally associated with the fact that the electrode needs to be immersed in the liquid and become contaminated, so that it will subsequently need to be washed. Furthermore, it is not possible to monitor the presence or absence of liquid by this technique in a hermetically closed consumable.

In the rest of the text, the term “capacitive detection” is intended to mean measurement of the capacitance variation of a capacitor formed by two electrodes or by one measurement electrode and a ground plane.

In a second field, direct contact between the electrode and the liquid is not necessary, these devices making it possible to monitor:
the presence of bubbles in these vessels, or
the filling level of said vessels.

In the case of monitoring the presence of bubbles which are present at the surface of the liquid, Document U.S. Pat. No. 4,312,341 proposes a device for monitoring the variation of liquid present in a container, in this case a tube, the device comprising a bubble detector. A light source is placed on one of the faces of the container, while a light detector is placed on the opposite face. In response to the intensity of the light signal coming from the light source, the detector generates an output signal proportional to the configuration of the luminosity present in the tube.

This detection method makes it possible to demonstrate the presence of bubbles, and therefore the presence of liquid, but it does not permit specific measurement of the volume of liquid present in the container.

Likewise, U.S. Pat. No. 4,371,786 describes a device for detecting the presence of bubbles in a liquid, the device comprising a radiation source and a radiation detector. The emitted rays have a configuration which then differs according to whether or not bubbles are present at the surface of the liquid. This invention is more particularly suitable for the medical field, when the intention is to detect and avoid the presence of bubbles in order to avoid any risk of embolism during the infusion of serum or blood into a patient.

The invention to which this patent relates, however, is not suitable for the measurement of a volume present in the container, but only for detection of the presence or absence of bubbles.

In the case of monitoring the filling level of said vessels, Document U.S. Pat. No. 5,017,909 is based on the same physical principle as that explained in Document U.S. Pat. No. 4,371,786, that is to say making it possible to measure a filling level of a liquid without contact of the electrode with this liquid. The measurement is thus carried out by determining a capacitance variation due to the difference in electrical permittivity between water and air. This measurement is carried out by the capacitive level measurement of liquids in vessels which are generally of large size, that is to say much more than 1 liter, and not closed.

As explained above, however, these methods of capacitive level detection of liquids often use vessels of large size as a container (vessels of several liters), since the capacitance variation is then large enough to be measured easily.

In turn, Patent Application GB-A-1,213,128 discloses fluidic level detection equipment using an electrical capacitance measurement. This detection is not very precise because it only makes it possible to detect a level predetermined as a function of the position at which the electrodes are fixed, and to trigger an alarm when the liquid meniscus reaches this threshold. This system is incapable of deducing the quantity of liquid present in each reaction well when the height of the meniscus varies between the two electrodes. Likewise, the equipment according to this application does not consist of two parts which enclose the container, a first part which includes at least one electrical transmission means and a second part which includes at least one electrical reception means, the transmission and/or reception means themselves including means which cancel the edge effects and the leakage currents, which means are particularly suitable for determining small volumes of liquid.

The application of such precise detection methods to very small volumes, in particular volumes of between a few microliters (μl) and a few milliliters (ml), is not found in the literature, which volumes are moreover commonly used in the technical field of biology, in particular. Furthermore, the measurement of a volume of a few microliters in a closed consumable, that is to say without contact with the instrument or instruments for measuring the volume, is definitively not described in these patents of the prior art.

It is an object of the present invention to resolve all of the drawbacks of the prior art by providing a method and a device for monitoring. the presence or absence of a volume of liquid in a container or for monitoring the filling of a container with a volume of liquid. This method and this device may be applied to containers which include many wells that receive a liquid, the wells each having a small volume like those which are used in the case of biological analysis cards, such as those described in Patent Application FR/98/11053 of the Applicant.

To that end, the present invention relates to a method for monitoring the presence or absence of a volume of liquid in a container or for monitoring the filling of a container with a volume of liquid, with at least one channel which leads to at least one well that receives some or all of the liquid passing through the container, the channel or channels and the well or wells constituting a fluidic network, the method consisting in:
filling some or all of the fluidic network,
detecting the capacitance, or any other parameter which is associated with it, such as the relative permittivity of the air present in the container or the oscillation frequency or the voltage, at each well, after the injection of said liquid,
determining the capacitive variation, or any other variation of a parameter which is associated with it, at each well, and
deducing the quantity of this liquid present in each reaction well.

According to another embodiment, a preliminary step is carried out in order to calibrate the capacitance, or any other parameter which is associated with it, with respect to a material with a known capacitance or any other parameter which is associated with it. This step may consist in detecting the capacitance, or any other parameter which is associated with it, at each well before the injection of the liquid. The calibration step may be carried out a single time on an empty card. The resulting values of frequencies are recorded and taken as a reference. The measurement values of each filled card are then compared with respect to the recorded values of frequency.

According to another embodiment, the fluidic network is closed after some or all of said network has been filled.

Also according to another embodiment, the detection of the capacitance, or of any other parameter which is associated with it, is carried out by accumulating the electrical charges between two electrodes, or between one electrode and a ground plane, on either side of each well without contact with the liquid volume aliquot present in each well.

According to another embodiment, each well has a volume less than or equal to 100 ml, preferably less than or equal to 10 ml, preferably less than or equal to 1 ml.

According to yet another embodiment, which makes it possible to count microorganisms present in the initial volume of liquid, by using at least two wells with different dimensions, each well containing the same medium, which is preferably specific to the microorganisms, making it possible to grow and optionally characterize the microorganisms to be counted, an additional step is carried out for processing the data relating:
to the liquid volume aliquot present in each well, and
to the number of positive or negative wells,
   in order to determine the initial concentration of microorganisms in said initial volume of liquid.

According to another embodiment, the capacitive variation is detected by:
a variation in the amplitude of a voltage by using an impedance bridge, for example, or
a variation in the frequency of a periodic AC signal (sinusoidal, triangular, square-wave, or any other periodic shapes) by using an oscillator, for example, or
examining an electrical circuit of the “trap” type, that is to say RLC, or
the variation in the maximum amplitude of the charge of a capacitor (consisting of the system) or the variation in the charging or discharging rate of the capacitor (to be measured) given that τ=R×C.

The capacitance of the “capacitor”, consisting of the card well surrounded by the two electrodes, may in general be detected by inserting this capacitor into any electronic circuit sensitive to a very small capacitance variation.

Also according to another embodiment, the steps of detecting the capacitance, or any other parameter which is associated with it, are carried out by pressurization between at least the two electrodes and/or between at least one electrode and the ground plane, with said two electrodes and/or said electrode and ground plane sandwiching at least one container.

The present invention also relates to a device for monitoring the presence or absence of a volume of liquid in a container or for monitoring the filling of a container with a volume of liquid, implementing a method as described above, with at least one channel which leads to at least one well that receives some or all of the liquid passing through the container, the channel or channels and the well or wells constituting a fluidic network. The device consists of two parts which enclose the container, a first part which includes at least one electrical transmission means and a second part which includes at least one electrical reception means, the transmission and/or reception means:
themselves including means which cancel the edge effects and the leakage currents, and
sandwiching each well with a volume less than or equal to 100 ml, preferably less than or equal to 10 ml, preferably less than or equal to 1 ml, without contact with the liquid volume aliquot possibly present in each well.

The ground plane may be defined as consisting of all the ground electrodes connected together and connected to the ground of the supply.

According to another embodiment, each transmission means includes an electrode, and each reception means includes an electrode which is grounded or preferably connected to a ground plane, and the means which cancel the edge effects and the leakage currents consist of at least one guard ring surrounding each electrode.

According to another embodiment, the transmission and reception means are pressed against each sandwiched well.

Also according to another embodiment, which makes it possible to count microorganisms present in the initial volume of liquid, by using at least two wells with different dimensions, each well containing the same medium, which is preferably. specific to the microorganisms, making it possible to grow and optionally characterize the microorganisms to be counted, the transmission and reception means associated with a well have dimensions and performances adapted to the dimension of said well.

According to another embodiment, the transmission and reception means are sandwiched between two substantially parallel containers and make it possible to carry out a method as described above, simultaneously or consecutively in said two containers.

According to yet another embodiment, the container or containers are in a vertical position.

Also according to another embodiment, the transmission and reception means are brought towards each other, while sandwiching at least one container, by using pressurization means, such as a pressure roller associated with a spring.

The present invention also relates to the implementation of the method as described above for monitoring the presence or absence of a volume of liquid in a container or monitoring the filling of a container with a volume of liquid, the volume of liquid being distributed between a plurality of wells, each well having a volume less than or equal to 1 ml, preferably less than or equal to 250 μl, preferably less than or equal to 25 μl, preferably less than or equal to 2.5 μl. These values are in accordance with the values tested below, which are 144 and 225 μl for the large wells, 30 and 22.5 μl for the medium wells and 2.875 and 2.25 μl for the small wells.

The present invention also relates to the implementation of the method as described above for counting microorganisms present in the initial volume of liquid, the volume of liquid being distributed between a plurality of wells, each well having a volume less than or equal to 1 ml, preferably less than or equal to 250 μl, preferably less than or equal to 25 μl, preferably less than or equal to 2.5 μl.

The present invention relates to the use of the device as described above for monitoring the presence or absence of a volume of liquid in a container or monitoring the filling of a container with a volume of liquid, the volume of liquid being distributed between a plurality of wells, each well having a volume less than or equal to 1 ml, preferably less than or equal to 250 μl, preferably less than or equal to 25 μl, preferably less than or equal to 2.5 μl.

The present invention also relates to the use of the device as described above for counting microorganisms present in the initial volume of liquid, the volume of liquid being distributed between a plurality of wells, each well having a volume less than or equal to 1 ml, preferably less than or equal to 250 μl, preferably less than or equal to 25 μl, preferably less than or equal to 2.5 μl.

The appended figures are given by way of explanatory example and imply no limitation. They allow the invention to be understood better.

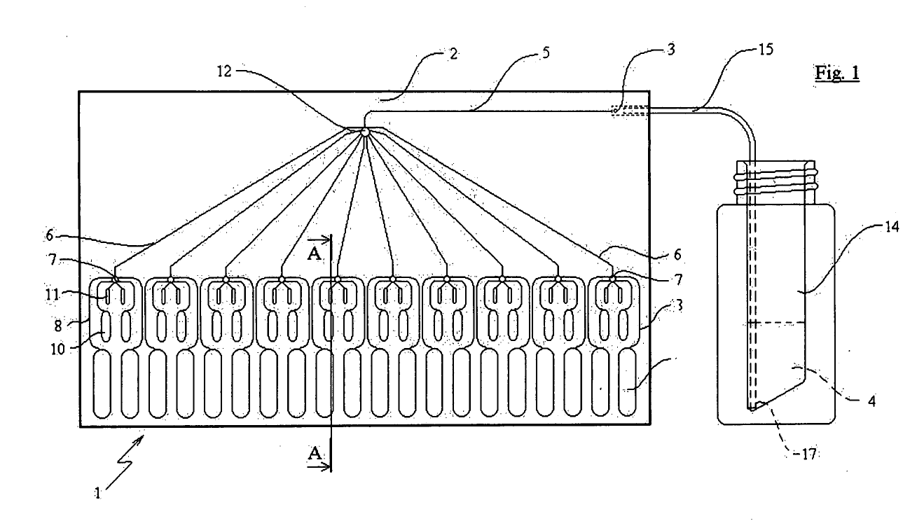

FIG. 1 demonstrates the context of using. a container according to the present invention, that is to say an analysis card. To that end, this figure represents a front view of a receptacle, which receives an initial volume of biological sample and is associated structurally and functionally with an analysis card, making it possible to count and characterize microorganisms present in this biological sample.

FIG. 2 represents a view in partial section on A-A of FIG. 1, with a magnification of two times compared with that of the first figure.

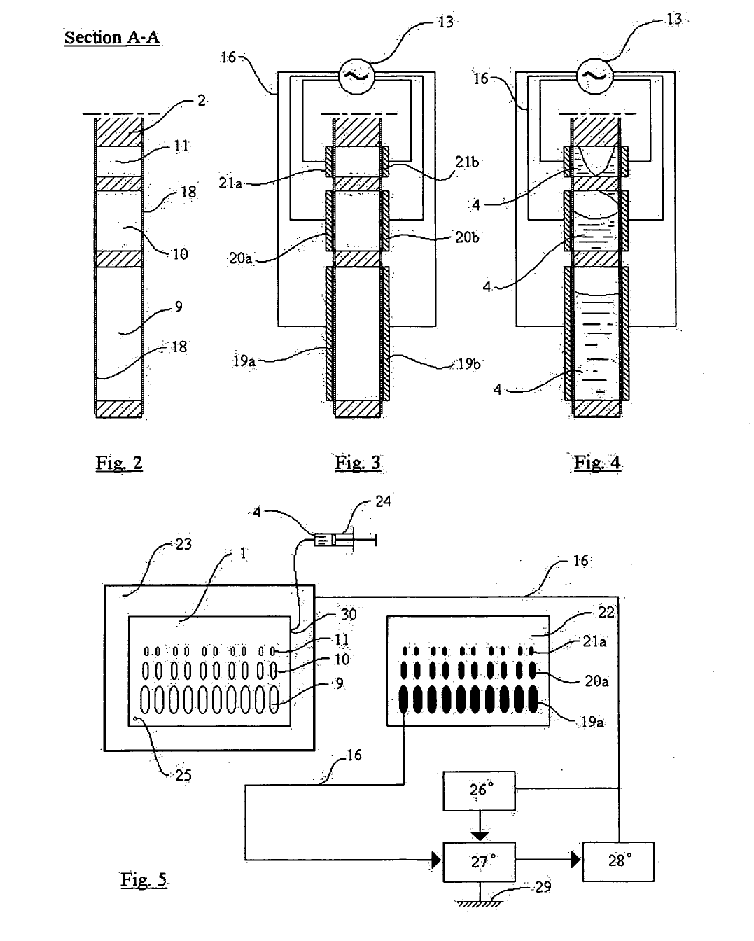

FIG. 3 represents a view which is identical to FIG. 2, but in which the analysis card is enclosed between detection electrodes constituting the capacitor, which is inserted into the electronic detection circuit, at each well, the wells being empty.

FIG. 4 represents an identical view to FIG. 3, the wells being partially filled.

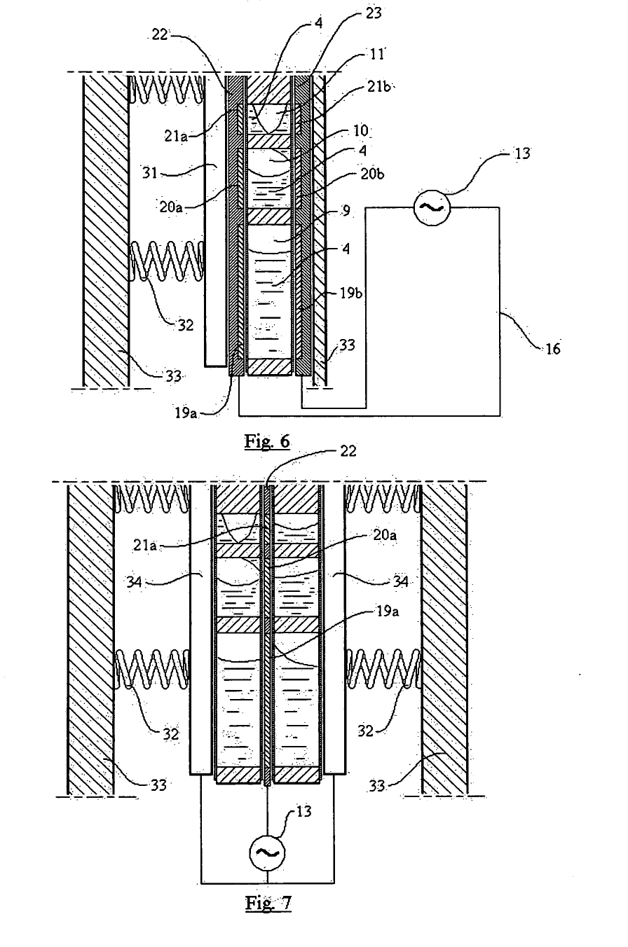

FIG. 5 represents a schematic overall view of the device for monitoring the presence or absence of the volume of liquid in the analysis card, or for monitoring the filling of said card with said volume of liquid, according to another embodiment of the present invention. The ground electrodes consist of a single ground plane in this case.

FIG. 6 represents a view of a monitoring device according to the invention, onto which the electrodes are applied with a constant pressure in order to promote the sensitivity and reproducibility of these measurements.

FIG. 7 represents a view of two monitoring devices according to the invention, onto which the electrodes are applied with a constant pressure in order to promote the sensitivity and reproducibility of these measurements.

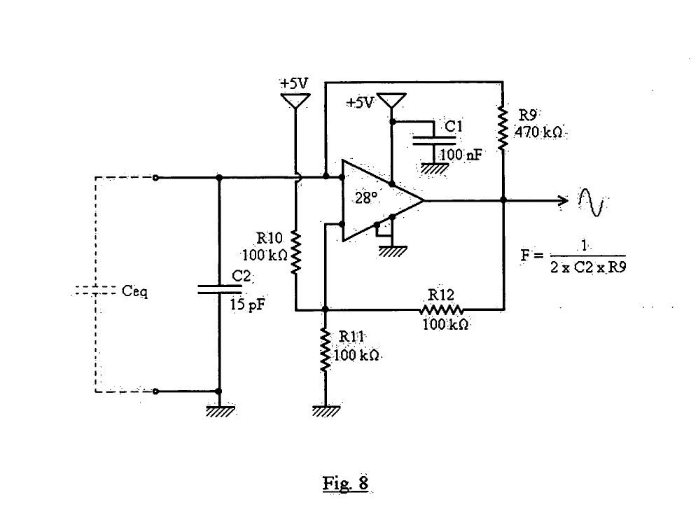

Lastly, FIG. 8 represents a view of an electronic layout of a monitoring device according to the invention.

Referring to FIG. 1, an analysis card 1 essentially consists of a single monobloc element consisting of the body 2 of the analysis card 1. This body 2 includes a zone 3 for injection of a biological liquid 4 to be analyzed, which is initially present in a receptacle 14. Such an analysis card may, for example, consist of a card such as described in Patent Application WO-A-00/12674 filed by the Applicant under the priority Sep. 1, 1998. The reader is invited to refer to this document for further information on this subject.

It is of course conceivable for the body 2 not to be monobloc, but to consist of a plurality of parts combined with one another by any fastening means of the prior art, such as adhesive, mechanical means (screw, bolt, rivet, elastic, etc.), welding. The injection zone 3 may furthermore be on any face of the body 2, whether on the side as represented in FIG. 1 or on the back, or else on another side wall. The card 1 is not filled directly with the sample 4 contained in the receptacle or tube 14, but is connected to this liquid 4 by using a flexible hose 15. The assembly formed in this way is ready to be placed in a vacuum chamber.

The feed channel 5 constitutes a chicane so that the injection zone 3 is positioned laterally with respect to the center of the body 2 of the card 1, that is to say it is extended inside said card 1 in the direction of one of the lateral surfaces of the latter.

The body 2 has a substantially parallelepipedal shape. This body 2 includes a certain number of grooves and cavities 9, 10 or 11 located inside it. These cavities may consist of through-holes, as is the case in FIGS. 2 to 4, or of blind holes according to an embodiment which is not represented in the figures.

The isolation of these grooves and these cavities from the exterior is carried out by applying a transparent adhesive film 18 which bonds to the surface of said body 2. It is advantageous to use the transparent adhesive film 18 because, after it has been pierced or taken off, it allows an operator to obtain direct access to the content of the cavities 9 to 11.

As regards the grooves, it will firstly be noted that there is a main feed channel 5 which is located downstream of the injection zone 3. Downstream of this channel 5, there is a well 12 which acts as a main distribution means for the biological liquid 4 to be analyzed. The main channel 5 is actually located between the injection zone 3 and the well 12. Similarly, there are secondary channels 6 which connect this well 12 to a set of wells 7, where each well 7 corresponds to one channel 6. These wells 7 act as distribution means. There are a certain number of terminal channels 8 at these wells 7, each channel 8 connecting one well 7 to one terminal analysis cavity 9, 10 or 11.

There are in fact three types of terminal analysis cavities in these figures, namely terminal analysis cavities 9 of large size, terminal analysis cavities 10 of medium size and, lastly, terminal analysis cavities 11 of small size.

It will therefore be understood clearly that the cavities located on the body 2 of the analysis card 1 are, on the one hand, the grooves which consist of the main channel 5, the secondary channels 6 and the terminal channels 8 and, on the other hand, the optionally blind holes which consist of the injection zone 3, the well 12, the wells 7 and all of the analysis cavities 9, 10 and 11.

The analysis cavities 9, 10 and 11 of different sizes make it possible to dispense with the need to carry out dilutions. As a function of the quantity of liquid 4 which is present in this analysis cavity 9, 10 or 11, there will therefore be a greater or lesser number of microorganisms. As a function of the culture medium to be added, it will therefore be possible to grow certain microorganisms which, as a function of their presence or their absence in each cavity 9, 10 or 11, will make it possible to obtain a modification of the optical density, the appearance or disappearance of fluorescence or of a color (chromogenic effect). This will make it possible to detect the presence or absence of microorganism(s) and/or to carry out quantification as well. Examples are explained in the Patent Application WO-A-00/12674 cited above.

There is in fact also a proportionality between the small cavities 11 and the large cavities 9, as well as with the medium cavities 10. The ratio existing between two cavities of different volumes, which are represented in the figures, is thus 1 to 10 between the cavities 10 and 9 and the cavities 11 and 10 and 1 to 100 between the cavities 11 and 9. This proportionality may also be encountered among all the channels 6 and 8.

A final point with regard to the structure of the analysis card 1 is that. none of the channels, whether they are main 5, secondary 6 or terminal 8, intersect with one another in order to avoid any contamination of a quantity of the sample located in one analysis cavity relative to a quantity of the sample located in another analysis cavity. To that end, the points of intersection of these various channels with one another are all formed by using wells which act not only as distribution means but also as a buffer volume preventing any contamination after the introduction of the sample 4.

The process of filling an analysis card 1, with reference to FIG. 1, therefore consists in:
generating a reduced pressure inside and/or in the vicinity of the card 1,
connecting the injection zone 3 of said card 1 to a volume of biological liquid 4 to be analyzed, which is present in the receptacle 14,
progressively breaking the reduced pressure so that, on the one hand, the biological liquid 4 is transferred into the analysis cavities 9, 10 and/or 11 and, on the other hand, air isolates the various fractions of the sample which come from said biological liquid 4, and which are present in each of said analysis cavities 9, 10 or 11.

Once the reduced pressure has been broken, air therefore comes in contact with the fractions of the biological sample 4, either in the terminal channels 8 according to FIG. 1 or in the analysis cavities 9, 10 and 11. This creates a physical separation between the bacteria. This, of course, can only be done if the volume of biological liquid 4 is in proportion with the total volume of the analysis cavities 9, 10 and 11, as well as optionally the volume of the terminal channel 8.

Instead of the air, any gaseous or liquid fluid which can fulfill this isolation function may be used. In this regard, care should be taken that the fluid is substantially inert with respect to the biological compounds contained in the sample, and immiscible with said sample. If the fluid is a liquid, for example an oil, it should have a lower density than the biological sample.

In this case, the card 1 should be kept substantially in a vertical position, as represented in FIGS. 1 to 4, the isolating fluid (not shown in the figures) being in a higher position than the biological sample previously contained in the receptacle 14, then in each cavity 9, 10 and 11.

It is, however, also possible to use other techniques for isolating or physically separating the cavities 9, 10 and 11. For instance, a gelling agent such as peptin may be added to the biological sample 4 and to the culture medium for the microbial growth. In this case, and when the gelling agent has fulfilled its function, there is no further need to keep the card 1 in a substantially vertical position.

Lastly, the isolation of said cavities 9, 10 and 11 may be carried out by obstructing the terminal channels 8. An adhesive may be used as isolating fluid, for example, or they may be obstructed by physical compression.

It is therefore clear that rigorous monitoring of the volume of sample 4 which is transferred from said receptacle 14 to the card 1 is of very great importance. It is the object of the present invention to ensure very rigorous monitoring of the volumes of liquid 4 which is distributed between the cavities 9, 10 and 11.

Another advantage consists in not keeping liquid 4, or keeping very little of it, inside the receptacle 14 after the transfer to said card 1. This was the object of a Patent Application filing FR01/09328 filed on Jul. 13, 2001 by the Applicant. The reader may refer to this document for further information on this subject.

The filling of the cavities 9, 10 and 11 moreover takes place simultaneously, at a given filling time, the ratio between the volume of liquid present in each size of cavity, small, medium or large, to the total volume of each size of cavity, respectively small, medium or large, being substantially constant for all of the cavities 9, 10 and 11 of the card 1.

Said card 1 according to FIG. 1 makes it possible to count the microorganisms contained in a biological sample 4, and it may be used in the field of food. In order to carry out this counting, the most probable number (MPN) of bacteria in a sample is calculated by a statistical method, explained by R. J. Parnow (1972). The occurrence probability is thus associated with each combination of positive(s). The method of J. C. De Man (1975) is used for this.

It is of course possible to have an analysis card which is similar from the point of view of the general concept but different in certain details, for example a card which includes four, five or more different sizes of cavities, this being in order to increase the investigation range of the card.

Of course, it is possible to perform a count of biological entities other than bacteria, such as nucleic acids (DNA or RNA), antibodies, antigens. The revealing may be carried out by anti-biological entity antibodies which are labeled.

If the total volume of the cavities 9, 10 and 11 is 4440 μl and the volume of the sample 4 is 2220 μl, the volume of the isolating liquid may be 2220 μl. It could be less if the intention is for there to be air in the card 1 once it has been sealed, or more if it desirable to ensure complete filling of the card with the sample 4 and the isolating liquid. In the latter case, this technique may be advantageous for the growth of anaerobic microorganisms.

In order first to determine fairly precisely the volume of the sample 4 which has been transferred into the card 1, it is therefore easy to understand that when the transfer of the sample 4 is taking place, the withdrawal first takes place at the level of said sample 4. It is therefore the level of the sample 4 which decreases until the free end 17 of the flexible hose 15 is in the isolating fluid. It is then that this fluid is secondly transferred to the card 1.

FIG. 2 represents a section on A-A of FIG. 1, in which the cavities 9, 10 and 11 consist of through-holes. These holes are delimited laterally by one or two films 18. This film or these films 18 consist of BOPP films (Biaxially Oriented PolyPropylene) or other films of the same kind, which are welded or adhesively bonded onto the body of the card 1, this body being inert with respect to the transferred liquids 4 and to the reactions into which they enter. This film or these films 18 may be present on all of the surface of the card 1, or on certain portions of said card 1.

The device according to the present invention is represented, for example, according to a first embodiment in FIG. 3. This FIG. 3 is identical to the section A-A of FIG. 2 as regards the card 1, which therefore consists of a body 2 defining a certain number of through-holes forming cavities of small size 11, medium size 10 and large size 9. All of these through-cavities are delimited laterally by the films 18. It can be seen that the cavities 9, 10 and 11 are not filled in this FIG. 3, although the device is schematically represented. In this context, it may be noted that the through-wells are in indirect contact via the film 18 with electrodes represented, on one hand on the left and, on the other hand, on the right of the figure.

The left-hand electrodes, which constitute the electrodes of the detection capacitor, may consist of large-area electrodes 19a corresponding to the terminal analysis cavities of large size 9, or else medium-area electrodes 20a corresponding to the terminal analysis cavities of medium size 10, or lastly small-area electrodes 21a corresponding to the terminal analysis cavities of small size 11. Likewise, the reception means consist of large-area electrodes 19b for the cavities 9, medium-area electrodes 20b for analysis cavities 10, and lastly small-area electrodes 21b for the cavities of small size 11. All these electrodes 19a, 20a and 21a, on the one hand, and 19b, 20b or 21b, on the other hand, are connected by electrical wire connections 16 to an electrical installation 13, as will be explained in the rest of the description.

FIG. 4 is absolutely identical to FIG. 3, with the only difference that the filling of the card 1 with a biological sample 4 has been carried out according to the embodiment represented in FIG. 1. In all of the cavities 9, 10 and 11 in this FIG. 4, there is therefore a presence of liquid 4 corresponding to aliquots of the biological sample 4 to be analyzed, which are distributed substantially proportionally as a function of the size of the cavities where they are received.

FIG. 5 describes another embodiment, the main difference of which is that all of the electrodes are grouped together irrespective of the face in question. As regards the first face, for instance, all of the electrodes 19a, 20a and 21a are brought to the level of a first part 22. This first part 22 is therefore provided with electrodes substantially in identical shape to the cross section of the cavities 9, 10 and 11 in the plane of the card 1, although the shape of these electrodes may be rectangular. These electrodes 19a, 20a and 21a are deposited by screen printing or consist of assembled metal plates. Each electrode is produced level with one of the faces of the first part 22, which part 22 may also carry the components including detection circuits duplicated identically for each electrode, the assembly being provided with a connector for being inserted into the electrical circuit (already mentioned in brief) by using the electrical installation 13 and the wire connections 16. In fact, the aforementioned electrodes 19a, 20a and 21a and the electronic components which operate them may be carried by the part 22, that is to say on one and the same card, on one or both faces of it 22, but they may also be carried by two different cards connected together by new wire connections 16.

The electronic detection circuits may be of various types, such as those mentioned below:
measurement based on the modification of the frequency of an oscillator by the input capacitance, consisting of the capacitor formed by the electrodes around the well,
measurement of the imbalance current of a bridge, in which case measurement of the current is generally preferable for the capacitance measurements owing to the impedance of the measuring equipment which is non-negligible,
measurement of a phase shift between two AC signals
measurement of the voltage across the terminals of a resonant circuit, such as the RLC trap circuit, quartz oscillator,
measurement of the output voltage of a lowpass filter of the third, fourth or higher order, the cutoff frequency of which is set to the empty capacitance of the well of the card.
   The capacitance variation of the cavities 9, 10 and 11 may in fact be detected according to two basic principles, either the variation in the amplitude of a voltage by using an impedance bridge or the variation in the frequency of a signal via an oscillator 27, as is clearly represented in FIG. 5.

In FIG. 5, it can be seen that the electrodes 19b, 20b or 21b are joined together to form a single ground plane 23, on which the card 1 is positioned. Irrespective of whether the ground plane 23 or all the electrodes of the first part 22 are involved, all of these elements are connected by wire connections 16 to an electrical circuit consisting of an electricity supply 26, an aforementioned oscillator 27 and an oscilloscope 28. The oscillator 27 is grounded by means of a grounding 29. In this figure, it can be seen that the wire connection 16 with the electrodes 19a, 20a and 21a is represented only with a single large-area electrode 19a. It is nevertheless clear that all of the electrodes are connected either the oscillator 27, as is the case in the figure, with a switching means for successively selecting the electrode and therefore the well to be monitored, or to as many oscillator as there are measurement electrodes.

In contrast to the embodiment of the card 1 according to FIGS. 1 to 4, where the liquid transfer utilizes evacuation of the entire fluidic network of said card 1 (which nevertheless keeps the same reference to avoid further complicating this description), the method of using the card 1 according to FIG. 5 is of a very different type since, for transferring the liquid sample 4, it uses a means of introducing said liquid 4 which consists of a syringe 24. This syringe 24 is connected by a flexible hose (not reference in this figure) to an orifice 30 for introducing the liquid 4 into the card 1. So that the air trapped in the fluidic network of the card 1 does not impede the introduction of the liquid 4, a vent 25 is made in the card 1, optionally through the film 18 if there is one, and acts as a means of evacuating the fluid present inside the card before the liquid 4 is introduced. This embodiment is particularly advantageous for filling the wells at will in order to check the measurement variation as a function of the filling level.

The last two FIGS. 6 to 7 return to the mode of using the card 1 according to FIGS. 1 to 4, that is to say with introduction of the liquid 4 after evacuation of the fluidic network of it 1. They are nevertheless two different embodiments of the device.

In FIG. 6, the device consists of a first part 22 and a second part 23 which carry all the electrodes, respectively of the first face 19a, 20a and 21a or of the second face 19b, 20b or 21b. These two parts 22 and 23 sandwich the card 1, while keeping each electrode 19a, 20a and 21a facing an identically sized electrode of the second face 19b, 20b or 21b. In this embodiment, it can be seen that the device consists essentially of the frame 33. The frame 33 therefore includes two vertical walls, right and left in this figure, the right-hand frame 33 acting as a support for the second part 23 that includes the electrodes 19b, 20b or 21b. Between the frame 33 located on the left and the first part 22 that includes the electrodes 19a, 20a and 21a, however, there is a pressure roller 31, which is itself joined to the frame 33 by using a spring 32. This configuration makes it possible to obtain pressure on the sandwich consisting of the first and second parts 22 and 23 and the card 1.

The main object of this configuration is to further improve the measurements which are carried out, as well as to avoid any subsequent analysis error. Of course, all of the electrodes are connected by wire connections 16 to the electrical installation 13 already described with reference to FIG. 5. Similarly as in this FIG. 5, it will be noted that not all of the wire connections 16 with all of the electrodes have been represented, in order to facilitate understanding of the figure.

This is also the case in FIG. 7, which presents another embodiment. This embodiment consists of a device including a frame 33, which is not in direct contact with the electrodes or with the cards 1. According to the embodiment which is represented, two cards 1 are filled and can be read. The frame 33, whether on the right or on the left, is thus combined with springs 32 which are themselves combined with a pressure roller 34 located on the left and with a pressure roller 34 located on the right. These pressure rollers 34 have characteristics of acting as a ground plane 34, that is to say they act as electrical reception means. They are each therefore in direct contact with a card 1, the two cards 1 being separated by a first part 22 where the electrodes 19a, 20a and 21a are identical for the analysis cards 1 located on either side. It is entirely conceivable to increase the size of this sandwich in order to alternately or simultaneously carry out analyses of at least three cards.

### EXAMPLE 1

### Evaluation of the Detection of the Filling of Wells having Small Volumes, which may be Different, Within A Card by Measuring the Capacitance Variation (Without Contact with the Liquid)

1) Protocol (Equipment):

Test card: polystyrene card with a well of about 4×3×4 mm (width×length×thickness),
Card 1 reference P11-20 (A03) according to Patent Application FR98/11053 filed on Sep. 1, 1998 by the Applicant,
Self-adhesive aluminum film (Scotch 425, 3M, reference 612975, supplier Radiospare, Beauvais, France),
Syringe 24, silicone tubes, connections, silicone paste for sealing,
Distilled water colored with Orange G,
Bracket for holding the card,
Fluck 85 Multimeter (Multimeter supplier Radiospare),
Fluck 96B ScopeMeter (Scopemeter or portable oscilloscope supplier Radiospare),
0 to 30 volt laboratory voltage supply (bioMérieux, Marcy l'Etoile, France, reference No 01809),
electronics card for (capacitive) detection, according to FIG. 8,
   2) Protocol (Method):
1. Calculation of the orders of magnitude,
2. Experiment on the test card made of high-impact polystyrene provided with a cavity measuring 4×3×4 mm, i.e. 48 μl,
3. Experiment on the card referenced 1,

For each assembly, the cavity under test is closed with an adhesive film. A tongue of self-adhesive aluminum film (forming the measurement electrode) is positioned on the adhesive film below the cavity under test.

For each experiment, the method consists in having a fluidic input and a fluidic output for filling and emptying the cavity under test at will. Each time it is filled and emptied, the variation in frequency or voltage resulting from the presence or absence of liquid is noted.

3) Results:

The main result to be obtained is to draw conclusions about the possibility of carrying out detection of empty or full wells by capacitance measurement.

A—Calculation of the Orders of Magnitude:

The calculations carried out make it possible to evaluate the capacitance variation of the capacitor formed by the well closed with the BOPP film and the two electrodes consisting of the two rectangles of self-adhesive aluminum film.

The equivalent capacitance Ceq of the test cell, taking the various layers into account, is given by the formula:

$$\frac{1}{Ceq} = \frac{1}{ɛ o} [ \frac{efilm}{ɛ film \cdot S} + \frac{ewell}{ɛ air \cdot S ( 1 - x ) + ɛ liq \cdot S \cdot x} + \frac{ewall}{ɛ wall \cdot S} ]$$

with: εfilm, εwall: relative permittivity of the BOPP (polypropylene) film and of the plastic (polystyrene),
εair: relative permittivity of air,
εliq: relative permittivity of the liquid,
S: surface area of the electrodes,
ewall: thickness of the plastic wall,
efilm: thickness of the BOPP film,
ewell: depth of the well, and
x: % filling of the well (0<x 21 1),
   knowing that:
relative permittivity of air=1,
relative permittivity of polypropylene and polystyrene˜2.
relative permittivity of pure water˜81, and
permittivity of free space εo=10⁻⁹/36π.

The permittivity of the tested liquids is close to 81. The literature gives values varying from 81 for pure water to 2 for mineral oils, passing through values lying between 40 and 20 for glycol and methyl alcohol, and the equivalent capacitance can be estimated from the above formula in both cases (empty well and full well):
empty well: x=0, whence Ceq˜6.15·10⁻¹⁴ F, and
full well: x=1, whence Ceq˜3.33·10⁻¹² F.

For the 48 μl test cell, the theoretical capacitance variation is ΔC˜3.27 pF. This is small, but should be detectable by a suitable electronic layout.

Table 1 below gives the theoretical values of capacitance variation for the wells of cards such as those represented in FIG. 1:

| Configuration | Well (144 μl) | Well (30 μl) | Well (2.875 μl) |
|---|---|---|---|
| Length of electrodes | 3.40 · 10⁻⁰² | 1.10 · 10⁻⁰² | 7.00 · 10⁻⁰³ |
| (m) | | | |
| Width of electrodes | 6.00 · 10⁻⁰³ | 5.00 · 10⁻⁰³ | 4.00 · 10⁻⁰³ |
| (m) | | | |
| Surface area of | 0.000204 | 0.000055 | 0.000028 |
| electrodes (m²) | | | |
| Distance between | 4.03 · 10⁻⁰³ | 4.03 · 10⁻⁰³ | 4.03 · 10⁻⁰³ |
| electrodes (m) | | | |
| BOPP film thickness | 2.50 · 10⁻⁰⁵ | 2.50 · 10⁻⁰⁵ | 2.50 · 10⁻⁰⁵ |
| (m) | | | |
| Wall depth (m) | 1.30 · 10⁻⁰³ | 1.30 · 10⁻⁰³ | 1.30 · 10⁻⁰³ |
| Well depth (m) | 2.70 · 10⁻⁰³ | 2.70 · 10⁻⁰³ | 2.70 · 10⁻⁰³ |
| Permittivity E1 (wall) | 2 | 2 | 2 |
| Permittivity E2 (film) | 2 | 2 | 2 |
| Permittivity E3 of the | 40 | 40 | 40 |
| liquid | | | |
| Vacuum permittivity | 8.84192 · 10⁻¹² | 8.84192 · 10⁻¹² | 8.84192 · 10⁻¹² |
| E0 | | | |
| Film capacitance | 1.443E · 10⁻¹⁰ | 3.89045 · 10⁻¹¹ | 1.98059 · 10⁻¹¹ |
| Wall capacitance | 2.775 · 10⁻¹² | 7.48163 · 10⁻¹³ | 3.80883 · 10⁻¹³ |
| Empty well | 5.36 · 10⁻¹³ | 1.45 · 10⁻¹³ | 7.36 · 10⁻¹⁴ |
| capacitance | | | |
| Filling (0 to 100%) | 100% | 100% | 100% |
| Filled well capacitance | 2.67222 · 10⁻¹¹ | 7.20453 · 10⁻¹² | 3.66776 · 10⁻¹² |
| Theoretical | 2.47 · 10⁻¹² | 6.66 · 10⁻¹³ | 3.39 · 10⁻¹³ |
| capacitance (F) | | | |
| Capacitance variation | 1.93 | 0.52 | 0.27 |
| (pF) | | | |

Table 1: Calculation of the Capacitance Variation between an Empty Well (Air) and a Full Well (Liquid)

The capacitance variations to be detected are therefore of the order of one picofarad, which requires very sensitive electronics which are properly protected from stray phenomena.

B—Experiment on a Single Test Cell Well:

B.1—Measurement by Direct Capacitance Detection:

Simple initial experiments of direct capacitive detection were carried out with a multimeter having the function of a capacimeter. The sensitivity of the multimeter did not permit a capacitance variation to be measured between an empty well and a full well. Direct measurement might be possible with a capacimeter having a sensitivity of the order of one picofarad.

B.2—Measurement of Variation in the Frequency with an Oscillator:

The assembly scheme corresponds substantially to FIGS. 6 and 7, but with a single well in the horizontal position, and it consists in testing a detection configuration with two electrodes (aluminum film adhesively bonded onto the polystyrene card or with a single electrode and a ground plane. The electrode is connected to the input of the oscillator circuit, and the output of the oscillator is connected to an oscilloscope for measuring frequency.

The dimensions of the well of the test cell are 4×3×4 mm (length×width×depth). The volume of the well being tested is 48 μl, which represents a volume fifteen times greater than the volume of the smallest well of the cards 1 (2.875 μl).

This experiment was carried out with an oscillator according to the assembly reproduced in FIG. 8.

The results of the measurements are presented in table 2 below:

| | | | Assembly with one | |
|---|---|---|---|---|
| | Assembly with two | | electrode and one | |
| | electrodes | | ground plane | |
| | Frequency | Frequency | Frequency | Frequency |
| | (kHz) with | (kHz) with | (kHz) with | (kHz) with |
| Measurement No | empty well | full well | empty well | full well |
| 1 | 70.36 | 69.07 | 65.86 | 64.66 |
| 2 | 70.10 | 69.11 | 65.65 | 64.61 |
| 3 | 69.58* | 69.12 | 65.74 | 64.57 |
| 4 | 69.79 | 69.07 | 65.60 | 64.43 |
| 5 | 69.72 | 69.06 | 65.48 | 64.42 |
| 6 | 69.84 | — | 65.36 | — |
| Average | 69.9 | 69.09 | 65.61 | 64.54 |
| CV | 0.26 | 0.024 | 0.16 | 0.097 |
| Δf | | 0.81 kHz | | 1.07 kHz |

*Presence of a bubble, well not correctly emptied. The oscillation frequency therefore lies between the two frequencies, on the one hand when empty (about 70 kHz), and on the other hand when full (about 69 kHz).

Table 2: Variation in the frequency between an empty well and a full well according to two different configurations by using the layout described in FIG. 8

The frequency variation Δf is about 0.8 kHz and 1 kHz, respectively, for the assembly with two electrodes and for the assembly with one electrode and a ground plane. The well is in a horizontal position for the experiments. The frequency in each state is furthermore stable and varies by about ±0.05 kHz in the configuration being tested.

These results show that the oscillator assembly are fairly sensitive, and stable enough to observe a frequency difference when the well is being filled. The results with the oscillator according to this assembly are good, because they are stable and have a fairly large frequency deviation. It is this type of oscillator which will be used subsequently.

Using a ground plane is more advantageous in terms of the measurements and in terms of easily implementing the monitoring of the volumes automatically. It is this type of configuration which will be used subsequently.

### EXAMPLE 2

### Experiment on the Analysis Card 1

1) Measurement of Variation in Oscillation Frequency for each Type of Well and for Various Liquids

The analysis card 1 being tested comprises well volumes (large, medium and small) which are respectively equal to 225 μl, 22.5 μl and 2.25 μl. The assembly is represented by FIG. 5.

The vertical position makes it possible to facilitate the filling of the well being tested. All the grounds are connected. The tests were carried out with three different liquids:
distilled water colored with orange G,
buffered peptonated water (42-042) Batch 740661901 (bioMérieux, Marcy l'Etoile, France), and
tryptone salt (42-076) Batch 740493801 (bioMérieux, Marcy l'Etoile, France).

A—Measurement on a 225 μl Well (Large):

Table 3 below gives the following results for the large wells and for the three different liquids mentioned above:

| | Colored distilled | | Buffered peptonated | | | |
|---|---|---|---|---|---|---|
| | water | | water | | Tryptone salt | |
| | Frequency | | Frequency | | Frequency | |
| | (kHz) | Frequency | (kHz) | Frequency | (kHz) | Frequency |
| | with | (kHz) | with | (kHz) | with | (kHz) |
| Measurement | empty | with full | empty | with full | empty | with full |
| No | well | well | well | well | well | well |
| 1 | 62.43 | 55.04 | 62.41 | 52.15 | 59.32 | 52.14 |
| 2 | 62.82 | 55.86 | 60.06 | 52.19 | 59.20 | 52.16 |
| 3 | 62.76 | 55.18 | 59.70 | 52.30 | 59.22 | 52.20 |
| 4 | 62.62 | 55.10 | 59.43 | 52.22 | 59.23 | 52.21 |
| 5 | 62.59 | 55.18 | 59.24 | 52.18 | 59.25 | 52.21 |
| 6 | 62.54 | — | 59.26 | — | 59.20 | — |
| Average | 62.63 | 55.3 | 60.02 | 52.21 | 59.24 | 52.18 |
| CV | 0.13 | 0.3 | 1.1 | 0.05 | 0.04 | 0.03 |
| Δf | | 7.33 kHz | | 7.81 kHz | | 7.06 kHz |

Table 3: Measurement of Variation in Oscillation Frequency for the 225 μl Well

Since the frequency variation has to be linear, there is therefore a frequency variation of about 35 hertz per microliter (Hz/μl).

B—Measurement on a 22.5 μl Well (Medium):

Table 4 below gives the following results for the medium wells and for the three different liquids mentioned above:

| | Colored distilled | | Buffered peptonated | | | |
|---|---|---|---|---|---|---|
| | water | | water | | Tryptone salt | |
| | Frequency | | Frequency | | Frequency | |
| | (kHz) | Frequency | (kHz) | Frequency | (kHz) | Frequency |
| | with | (kHz) | with | (kHz) | with | (kHz) |
| Measurement | empty | with full | empty | with full | empty | with full |
| No | well | well | well | well | well | well |
| 1 | 70.84 | 67.07 | 70.94 | 67.15 | 70.56 | 66.70 |
| 2 | 70.74 | 67.08 | 70.98 | 67.14 | 70.57 | 66.53 |
| 3 | 70.83 | 67.08 | 70.99 | 67.44* | 70.48 | 66.48 |
| 4 | 70.79 | 67.23* | 70.96 | 67.19 | 70.48 | 67.67 |
| 5 | 70.82 | 67.48* | 70.93 | 67.17 | 70.79 | 67.12 |
| 6 | 70.83 | — | 70.95 | — | 70.72 | — |
| Average | 70.81 | 67.19 | 70.96 | 67.22 | 70.6 | 66.9 |
| CV | 0.034 | 0.157 | 0.02 | 0.11 | 0.12 | 0.45 |
| Δf | | 3.62 kHz | | 3.74 kHz | | 3.7 kHz |

*Presence of a bubble, well not completely emptied.

Table 4: Measurement of Variation in Oscillation Frequency for the 22.5 μl Well

Since the frequency variation has to be linear, there is therefore a frequency variation of about 165 Hz/μl.

C—Measurement on a 2.25 μl Well (Small):

Table 5 below gives the following results for the small wells and for the three different liquids mentioned above:

| | Colored distilled | | Buffered peptonated | | | |
|---|---|---|---|---|---|---|
| | water | | water | | Tryptone salt | |
| | Frequency | | Frequency | | Frequency | |
| | (kHz) | Frequency | (kHz) | Frequency | (kHz) | Frequency |
| | with | (kHz) | with | (kHz) | with | (kHz) |
| Measurement | empty | with full | empty | with full | empty | with full |
| No | well | well | well | well | well | well |
| 1 | 70.76 | 69.29 | 70.48 | 69.21 | 70.32 | 69.08 |
| 2 | 70.77 | 69.37 | 70.43 | 69.17 | 70.33 | 69.10 |
| 3 | 70.46 | 69.32 | 70.38 | 69.26 | 70.47 | 69.14 |
| 4 | 70.44 | 69.29 | 70.34 | 68.89 | 70.29 | 69.10 |
| 5 | 70.40 | 69.33 | 70.32 | 69.08 | 70.47 | 69.12 |
| 6 | 70.46 | — | 70.33 | — | 70.50 | — |
| Average | 70.55 | 69.32 | 70.38 | 69.12 | 70.4 | 69.11 |
| CV | 0.15 | 0.03 | 0.06 | 0.13 | 0.08 | 0.02 |
| Δf | | 1.23 kHz | | 1.26 kHz | | 1.29 kHz |

Table 5: Measurement of Variation in Oscillation Frequency for the 2.25 μl Well

Since the frequency variation has to be linear, there is therefore a frequency variation of about 555 Hz/μl.

2) Measurement of the Stability of the Oscillation Frequency for each the Wells of Medium Size:

Table 6 below gives the following results for the wells of medium size. The same stability was observed for the wells of other sizes:

| Time | Frequency (kHz) | Frequency (kHz) |
|---|---|---|
| (min) | empty well | empty well |
| 0 min | 70.72 | 67.46 |
| 1 min | 70.77 | 67.49 |
| 2 min | 70.81 | 67.53 |
| 3 min | 70.83 | 67.55 |
| 4 min | 70.86 | 67.56 |
| 5 min | 70.89 | 67.60 |

Table 6: Measurement of the Frequency Drift of the Oscillator for the 22.5 μl Well

During the measurement of the frequency variation of the oscillator after 5 minutes, when the well is full or empty, a slight variation is observed over time. The stability of the oscillation frequency is nevertheless sufficient to discriminate between the two states.

### EXAMPLE 3

### Configuration of the Device for Monitoring the Presence or Absence of a Volume of Liquid or the Filling of an Analysis Card with a Liquid

The monitoring device for filling is schematically represented in the figures. It may, however, be formed by assembling two cards:
one card carrying the electrodes: This card is provided with rectangular electrodes (deposited by screen printing or consisting of metal plates, for example). Each electrode is connected to the opposite face of the card, then to the “component” card. The reference 22 in FIG. 5 fully reflects this idea. This card may be fitted elastically (springs) on the “component” card in order to permit good contact between the electrodes and the film of the analysis card 1. Such elastic layouts are described clearly in FIGS. 6 and 7.
one card carrying the components: if one face of said card comprises thirty wells, there may be thirty identical circuits fitted as SMCs, that is to say as surface-mount components, for serialization. This makes it possible to limit the stray capacitances and inductances and to stabilize the influence of these stray signals. The output signals (connector) are sent to an acquisition and processing module integrated in a computer.

If the wiring of the component card can be carried out on just one face, the two cards may be reduced to a single card (an electrode face and a component face). The “electrode” card may, for instance, have about the same dimensions as the card 1 to be monitored, i.e. about 90×60 mm. The “component” card, which may be larger, can be fixed onto the Electrode card so as to minimize the connection wires.

### EXAMPLE 4

### Monitoring Procedure

The volume monitoring procedure may be carried out with the following successive steps:
CALIBRATION: Measurement of the output values (frequency or voltage) of each detection circuit on the analysis card by varying the filling level.
MONITORING: Measurement of the output values (frequency or voltage) of each detection circuit after filling said card.
TEST: Comparison of the measured frequency variation with the frequency variation obtained from the calibration, which gives the filling level.
RESULTS: Rejection if the filling level less than 90%, for example, and acceptance if this level is higher than this percentage.

If the measurement is robust enough, the calibration of the monitoring cards may be carried out at a regular interval, but without the need to perform on a control reference before each measurement. The filling may also be monitored in real time in the filling jar.

### EXAMPLE 5

### Assembly to Monitor the Filling of the Cards in the Monitoring Device

A particularly advantageous version is presented in FIG. 6. Two methods according to the invention may be carried out successively, for example, after filling. For instance, the cards may be monitored two times, without having to pivot the card but simply by sliding it. Said card is pushed into the monitoring unit of the first face (configuration in FIG. 5) then into the monitoring unit of the second face (reverse configuration of the electrodes and of the ground plane than in FIG. 6).

### EXAMPLE 6

### Example Showing Detection of a Variation of Filling in a Well

1) Goal and Principle:

The goal is to produce an electronic card for volume monitoring in the wells of the analysis card 1. This electronic card, which was taken as a prototype for feasibility, consists of a card of epof material oxy material, on one side of which the detection electrodes which form one of the two electrodes of the measured capacitor are etched (one per well, i.e. thirty in all), and on the other side the SMC components (an operational amplifier and a resistor) for both injecting the sinusoidal input and output signal and taking the output signal, without experiencing electromagnetic perturbations, via the connection wires to the demodulation electronics (high input impedance of the amplifier).

As a reminder, the principle consists in detecting the capacitance variation of the capacitor formed by the measurement electrode (on the monitoring card) and by the ground plane. Since the permittivity of the liquids (εabout 40 to 81) is very different from that of air (ε-1), filling the well which constitutes the dielectric of the capacitor, leads to a capacitance variation because, to first order: C=εₒεᵣ.S/d.

Moreover, in order to avoid the leakage currents and the effect of all the stray capacitances in the vicinity, each electrode is furthermore provided with a guard electrode. This also makes it possible to greatly limit the edge effects of the measurement electrodes.

FIGS. 5, and more precisely 8, give the assembly scheme of the test bench. The electronic monitoring card is simply applied onto the analysis card, and is held secure by a clip. There are positioning references on the analysis card so that the electrodes, which are produced by screen printing, can be positioned as perfectly as possible on the wells of said card.

2) Equipment and Method:

2.1 Equipment:
Analysis card described in 1),
Electronic monitoring card, see FIGS. 5, and more precisely 8,
Philips PM3335 oscilloscope, 50 MHz (Hollande),
HP 33120A function generator,
Kd scientific syringe driver (Bioblock, France),
Laboratory supply (bioMérieux),
Syringe (Terumo 1 ml or Hamilton 50 μl) (Bioblock), silicone tubes, connections,
Distilled water colored with Orange G, buffered peptonated water Batch 42042 and 42043,
Iron metal plate for the ground plane,
Bracket for holding the analysis card.

2.2 Method:

This consists in:
assembling the test bench as described in FIGS. 5, and more precisely 8, with all of the grounds being connected,
filling the syringe with the liquid to be injected into the well,
fitting the analysis card on the support in order to permit the injection of liquid directly into a single well (small, medium or large), and connecting the injection tubing to the syringe placed on the syringe driver,
applying the electronic monitoring card onto the analysis card, while aligning the two top-left corners of the cards and fixing the assembly by pressure from a nonmetallic clip. The electrodes, which are produced by screen printing, need to be in intimate contact with the BOPP film of the analysis card,
adjusting the function generator in order to inject a sinusoidal input signal Vi, F=1 kHz and Viₚₚ (peak-to-peak input voltage)=1 V,
displaying the (sinusoidal) output signal Vo of the oscillator on the oscilloscope,
the well being empty, finding the resonant frequency (maximum amplitude) of the signal Vo (output voltage) by varying the frequency of the signal Vi and noting the frequency value,
injecting a volume of liquid into the well,
finding the new resonant frequency, which should be lower than the previous frequency, again by finding the amplitude maximum of Vo,
repeating the previous two steps until the well is completely filled, and
establishing the curve of Fr (Frequency at resonance) as a function of the volume over several experiments (volume injected) in order to check that the detection is reproducible.
   3) Results:

We worked with wells of large size, the temperature of the laboratory was 22° C. and the injected liquid was buffered peptonated water. The results obtained are presented in Table 7 below.

| | Resonant frequency (kHz) | | | | | |
|---|---|---|---|---|---|---|
| Volume (μl) | Test 1 | Test 2 | Test 3 | Test 4 | Test 5 | Test 6 |
| 0 | 35 | 30.8 | 30.2 | 33.2 | 34.8 | 30.4 |
| 40 | 29.6 | 29.5 | 29.2 | 28.8 | 29.6 | 28.9 |
| 80 | 28.2 | 28.3 | 27.9 | 27.5 | 28.2 | 27.8 |
| 120 | 27.4 | 27.4 | 26.8 | 26.5 | 27.3 | 27.2 |
| 160 | 27.2 | 26.6 | 26.2 | 25.7 | 26.5 | 26.5 |
| 200 | 25.8 | 25.9 | 25.7 | 25.3 | 26 | 25.7 |
| 240 | 25.3 | 25.5 | 24.8 | 24.6 | 25.3 | 25.4 |
| 280 | 24.3 | 24.6 | 24.1 | 22.7 | 24.5 | 24.5 |
| 320 | 23.7 | 22.9 | 23.1 | 22.8 | 23 | 22.8 |

Table 7: variation in the resonant frequency of the oscillator (demodulation) as a function of the filling level the large well.
Conclusion

The measurements carried out show that it is possible to detect capacitance variation without contact with the liquid in order to detect the presence or absence of a liquid in a well.

The following variations in oscillation frequency between an empty well and a full well were furthermore obtained on the analysis card 1:

225 μl well: ΔF˜7.5 kHz

22.5 μl well: ΔF˜3.7 kHz

2.25 μl well: ΔF˜1.25 kHz

For the three liquids which were tested (two of which are the dilation buffers of the stock media), detection is possible in all three wells and the measured values of DF are similar.

References
1. Container or analysis card
2. Body of the analysis card 1
3. Zone for injection into the card 1
4. Liquid or biological sample to be analyzed
5. Main feed channel
6. Secondary channels
7. Well corresponding to each channel 6, or distribution means
8. Terminal channels
9. Terminal analysis cavities, of large size, corresponding to a channel 8
10. Terminal analysis cavities, of medium size, corresponding to a channel 8
11. Terminal analysis cavities, of small size, corresponding to a channel 8
12. Well or main distribution means
13. Electrical installation associated with the electrodes 19, 20 and 21
14. Receptacle containing the sample 4
15. Transfer means or hose connecting the injection zone 3 to the tube 14
16. Electrical wire connection
17. Free end of the hose 15
18. Film fixed on the card 1
19a. Electrical transmission means or large-area electrodes corresponding to the terminal analysis cavities 9 of large size
20a. Electrical transmission means or medium-area electrodes corresponding to the terminal analysis cavities 10 of medium size
21a. Electrical transmission means or small-area electrodes corresponding to the terminal analysis cavities 11 of small size
19b. Electrical reception means or large-area electrodes corresponding to the terminal analysis cavities 9 of large size
20b. Electrical reception means or medium-area electrodes corresponding to the terminal analysis cavities 10 of medium size
21b. Electrical reception means or small-area electrodes corresponding to the terminal analysis cavities 11 of small size
22. First part including the electrical transmission means 19a, 20a and 21a
23. Ground plane or second part including the electrical reception means 19b, 20b and 21b
24. Means for introducing the liquid 4 into the analysis card 1, or syringe
25. Means for discharging a liquid from the analysis card 1, or vent
26. Electricity supply
27. Oscillator
28. Oscilloscope or scopemeter
29. Grounding
30. Orifice for introducing the liquid 4 from the means 24 into the card 1
31. Pressure roller
32. Spring
33. Frame of the device
34. Ground plane acting as pressure roller

1. A method for monitoring the presence or absence of a volume of liquid (4) in a container (1) or for monitoring the filling of a container (1) with a volume of liquid (4), with at least one channel which leads to at least one well that receives some or all of the liquid (4) passing through the container (1), the channel or channels and the well or wells constituting a fluidic network, characterized in that it consists in:
filling some or all of the fluidic network,
detecting the capacitance, or any other parameter which is associated with it, such as the relative permittivity of the air present in the container (1) or the oscillation frequency or the voltage, at each well, after the injection of said liquid (4),
determining the capacitive variation, or any other variation of a parameter which is associated with it, at each well, and
deducing the quantity of this liquid (4) present in each reaction well.

2. The method as claimed in claim 1, characterized in that a preliminary step is carried out in order to calibrate the capacitance, or any other parameter which is associated with it, with respect to a material with a known capacitance, or any other parameter which is associated with it, such as detecting the capacitance, or any other parameter which is associated with it, at each well before the injection of the liquid (4).

3. The method as claimed in claim 1, characterized in that the fluidic network is closed after some or all of said network has been filled.

4. The method as claimed in claim 1, characterized in that the detection of the capacitance, or of any other parameter which is associated with it, is carried out by accumulating the electrical charges between two electrodes, or between one electrode and a ground plane, on either side of each well without contact with the liquid volume aliquot (4) present in each well.

5. The method as claimed in claim 1, characterized in that each well has a volume less than or equal to 100 ml, preferably less than or equal to 10 ml, preferably less than or equal to 1 ml.

6. The method as claimed in claim 1 for counting microorganisms present in the initial volume of liquid (4), by using at least two wells with different dimensions, each well containing the same medium, which is preferably specific to the microorganisms, making it possible to grow and optionally characterize the microorganisms to be counted, characterized in that an additional step is carried out for processing the data relating:
to the liquid volume aliquot (4) present in each well, and
to the number of positive or negative wells,
in order to determine the initial concentration of microorganisms in said initial volume of liquid (4).

7. The method as claimed in claim 1, characterized in that the capacitive variation is detected by:
a variation in the amplitude of a voltage by using an impedance bridge, for example, or
a variation in the frequency of a signal (sinusoidal, triangular, square-wave, or any other periodic shapes) by using an oscillator, for example, or
examining an electrical circuit of the “trap” type, that is to say RLC, or
the variation in the maximum amplitude of the charge of a capacitor (consisting of the system) or the variation in the charging or discharging rate of the capacitor (to be measured) given that τ=R×C.

8. The method as claimed in claim 4, characterized in that the steps of detecting the capacitance, or any other parameter which is associated with it, are carried out by pressurization between at least the two electrodes and/or between at least one electrode and the ground plane, with said two electrodes and/or said electrode and ground plane sandwiching at least one container (1).

9. A device for monitoring the presence or absence of a volume of liquid (4) in a container (1) or for monitoring the filling of a container (1) with a volume of liquid (4), implementing the method as claimed in claim 1, with at least one channel which leads to at least one well that receives some or all of the liquid (4) passing through the container (1), the channel or channels and the well or wells constituting a fluidic network, characterized in that it consists of two parts which enclose the container, a first part which includes at least one electrical transmission means and a second part which includes at least one electrical reception means, the transmission and/or reception means:
themselves including means which cancel the edge effects and the leakage currents, and
sandwiching each well with a volume less than or equal to 100 ml, preferably less than or equal to 10 ml, preferably less than or equal to 1 ml, without contact with the liquid volume aliquot (4) possibly present in each well.

10. The device as claimed in claim 9, characterized in that each transmission means includes an electrode, and each reception means includes an electrode which is grounded or preferably connected to a ground plane, and in that the means which cancel the edge effects and the leakage currents consist of at least one guard ring surrounding each electrode.

11. The device as claimed in claim 9, characterized in that the transmission and reception means are pressed against each sandwiched well.

12. The device as claimed in claim 9 for counting microorganisms present in the initial volume of liquid (4), by using at least two wells with different dimensions, each well containing the same medium, which is preferably specific to the microorganisms, making it possible to grow and optionally characterize the microorganisms to be counted, characterized in that the transmission and reception means associated with a well have dimensions and performances adapted to the dimension of said well.

13. The device as claimed in claim 9, characterized in that the transmission and reception means are sandwiched between two substantially parallel containers (1) and make it possible to carry out a method as claimed in any one of claims 1 to 7, simultaneously or consecutively in said two containers (1).

14. The device as claimed in claim 9, characterized in that the container or containers (1) are in a vertical position.

15. The device as claimed in claim 9, characterized in that the transmission and reception means are brought towards each other, while sandwiching at least one container (1), by using pressurization means (31 or 34, 32 and 33), such as a pressure roller (31 or 34) associated with a spring (32).

16. The implementation of the method as claimed in claim 1 for monitoring the presence or absence of a volume of liquid (4) in a container (1) or monitoring the filling of a container (1) with a volume of liquid (4), the volume of liquid (4) being distributed between a plurality of wells (9, 10 and 11), each well (9, 10 and 11) having a volume less than or equal to 1 ml, preferably less than or equal to 250 μl, preferably less than or equal to 25 μl, preferably less than or equal to 2.5 μl.

17. The implementation of the method as claimed in claim 6 for counting microorganisms present in the volume of liquid (4), the volume of liquid (4) being distributed between a plurality of wells (9, 10 and 11), each well (9, 10 and 11) having a volume less than or equal to 1 ml, preferably less than or equal to 250 μl, preferably less than or equal to 25 μl, preferably less than or equal to 2.5 μl.

18. The use of the device as claimed in claim 9 for monitoring the presence or absence of a volume of liquid (4) in a container (1) or monitoring the filling of a container (1) with a volume of liquid (4), the volume of liquid (4) being distributed between a plurality of wells (9, 10 and 11), each well (9, 10 and 11) having a volume less than or equal to 1 ml, preferably less than or equal to 250 μl, preferably less than or equal to 25 μl, preferably less than or equal to 2.5 μl.

19. The use of the device as claimed in claim 12 for counting microorganisms present in the initial volume of liquid (4), the volume of liquid (4) being distributed between a plurality of wells (9, 10 and 11), each well (9, 10 and 11) having a volume less than or equal to 1 ml, preferably less than or equal to 250 μl, preferably less than or equal to 25 μl, preferably less than or equal to 2.5 μl.
